(19) United States
(12) Patent Application Publication
(10) Pub. No.: US 2005/0032066 A1
(43) Pub. Date: Feb. 10, 2005

(54) Method for assessing risk of diseases with multiple contributing factors

(76) Inventors: Chew Kiat Heng, Singapore (SG); Javier F. Cabrera, Piscataway, NJ (US); Chen Khong Tham, Singapore (SG)

Correspondence Address: Killworth, Gottman, Hagan & Schaeff, L.L.P., Suite 500, One Dayton Centre, Dayton, OH, 45402-2023, US

(21) Appl. No.: 10/634,145

(22) Filed: Aug. 4, 2003

(51) Int. Cl. C12Q 1/68; G06F 19/00; G01N 33/48; G01N 33/50

(52) U.S. Cl. 435/6; 702/20

(57) ABSTRACT

Methods for determining statistical models for predicting disease risks of a population are provided. Two types of data associated with members of the population are collected. The data may include both genetic and non-genetic types of data. A candidate statistical model is selected for calculating the disease risk. The model has a plurality of parameters and is a function of only one of the two types of data. A data weight is determined for each member of the population. Members having like data of the other type have like weights. The parameters of the model are optimized by fitting the collected data to the model taking into account of the weights.

### FIELD OF THE INVENTION

The present invention relates generally to assessing disease risks, and more particularly to determining statistical models for assessing disease risks affected by multiple factors.

### BACKGROUND OF THE INVENTION

Predicting disease risk is important in disease prevention. A disease risk is the probability that an individual will develop the disease in a given period of time. Disease risk may depend on multiple risk factors including both genetic factors and non-genetic factors. Disease risk is typically predicted using statistical risk prediction models determined from statistical analysis of sample data indicative of the risk factors from a given population.

Genetic factors, as used herein, refer to factors that are measured by genotyping and may include an individual's genotype profile, particularly polymorphic profile. Polymorphism refers to the co-existence of multiple forms of a genetic sequence in a population. The most common polymorphism is Single Nucleotide Polymorphism (“SNP”), a small genetic variation within a person's DNA sequence. SNPs occur frequently throughout the human genome. They are often associated with, or located near a gene found to be associated with, a certain disease. Thus, SNPs are genetic markers indicative of genetic disease risk factors as they mark the existence and locations of genes that render an individual susceptible to a disease. Since SNPs tend to be genetically stable, they are excellent genetic markers of diseases. For examples of known methods of assessing disease risks based on genetic markers see U.S. Pat. No. 6,162,604 to Jacob; U.S. Pat. No. 4,801,531 to Frossard; and U.S. Pat. No. 5,912,127 to Narod and Phelan.

Non-genetic factors refer to factors that are not measured by genotyping, such as age, sex, race, family history, height and weight, as well as environmental factors, such as smoking habit and living conditions.

As is known, a cumulative disease risk, denoted as R(t), can be calculated from a hazard function (h(t)),

$$R ( t ) = 1 - exp \{ - {\int }_{0}^{t} h ( u ) ⅆ u \} .$$

In a Cox proportional hazard regression model (“Cox model”), h(t) is assumed to be proportional to a base hazard (h₀(t)):

$$h ( t ) = {h}_{0} ( t ) exp ( {\sum }_{1}^{n} {β}_{i} {x}_{i} )$$

where βᵢ are empirical coefficients and xᵢ are variables indicative of risk factors. By fitting data collected from a population to the model, the coefficients βᵢ can be optimised and the optimised model can then be used to calculate the risk that a member of the population will have the disease at a given time.

However, different types of risk factors affect disease risks in different ways, yet they are often interdependent and may collaborate or interfere with each other. Therefore, it is often difficult to unravel the interplay between them by analyzing their effects on disease risks simultaneously. Conventionally, the effects of genetic and non-genetic factors are analyzed separately. For example, many known disease risk prediction methods would simply exclude a genetic factor if its effects appear to be correlated to environmental factors. This approach ignores the interplay completely and may lead to incorrect prediction. It is possible to analyze the effects of non-genetic factors for each possible combination of genetic markers, thus taking into account of both types of factors. See for example Pharoah et al. “Polygenic susceptibility to breast cancer and implications for prevention,” Nature Genetics 31:33-36 (2002). However, this approach is impractical if the number of genetic markers is large, thus resulting in an even larger number of possible combinations. For example, complex diseases, which have complex modes of inheritance, are usually affected by a large number of genetic risk factors as well as non-genetic factors. When the number of risk factors is large, the computation resources required often exceed what is available or practical because statistical analysis of the sample data is computation intensive.

Consequently, there are currently no satisfactory disease risk assessment methods that simultaneously and accurately take into account of a large number of both genetic and non-genetic risk factors.

In addition, known disease risk prediction methods often do not analyze available sample data properly and efficiently. For example, known risk assessment methods classify individuals providing the sample data as sick subjects (cases) or healthy subjects (controls). However, some subjects are inevitably misclassified because some control subjects would inevitably develop the disease given time. Further, known methods rely on the assumption that the subjects are truly representative of the population. Often, this assumption is incorrect because the sample size is not large enough and the subject selection is not truly random due to cost and other reasons. The problem is exacerbated when samples with missing values have to be discarded, which is a common practice in the field of disease risk studies. Although missing values may be imputed, existing imputation techniques require computation-intensive calculations and are not practical when the data size and the number of risk factors are large.

There is thus need for a disease risk assessment method that can effectively and efficiently analyze all available data indicative of a large number of risk factors, including both genetic and non-genetic risk factors.

### SUMMARY OF THE INVENTION

According to an aspect of the invention, there is provided a method of determining a statistical model for predicting disease risk for a member of a population. The method includes: collecting a plurality of sets of data, each of the sets of data associated with one member of the population, and including data of a first type, data of a second type, and an indicator of disease status of the one member associated with the set; selecting a candidate statistical model for calculating the disease risk as a function of data of the first type, the candidate model dependent on a plurality of parameters; determining a plurality of weights, each one of the weights associated with one of the sets of data and indicating a statistical significance of the one of the sets of data, wherein weights associated with sets of the data having like data of the second type are the same; and optimizing the parameters of the candidate model by fitting the plurality of sets of data to the candidate model, taking into account the weights.

According to another aspect of the invention, there is provided a computing system adapted to perform this method.

According to yet another aspect of the invention, there is provided a computer readable medium embedded thereon computer executable instructions, which when executed by a computer causes the computer to determine a statistical model for predicting disease risk for a member of a population by collecting a plurality of sets of data, each of the sets of data associated with one member of the population, and comprising data of a first type, data of a second type, and an indicator of disease status of the one member associated with the set; selecting a candidate statistical model for calculating the disease risk as a function of data of the first type, the candidate model dependent on a plurality of parameters; determining a plurality of weights, each one of the weights associated with one of the sets of data and indicating a statistical significance of the one of the sets of data, wherein weights associated with sets of the data having like data of the second type are the same; and optimizing the parameters of the candidate model by fitting the plurality of sets of data to the candidate model, taking into account the weights.

According to still another aspect of the invention, there is provided a method of imputing missing data indicative of a plurality of factors, comprising: determining a correlation between the plurality of factors; grouping the factors into batches such that all factors in each the batch are correlated; and imputing missing data for factors in one the batch at a time.

According to yet another aspect of the invention, there is provided a method of grouping a plurality of data sets into groups, comprising dividing the plurality of data sets into two or more groups depending on data indicative of a factor of a first type in each of the data sets; determining if a criterion is met after the dividing, the criterion is evaluated based on data of a second type in each of the data sets; and when the criterion is not met, regrouping the plurality of data sets back into one group.

According to still another aspect of the invention, there is provided a method of weighing a plurality of data sets, each one of the data sets associated with a member of a population, comprising weighing each set of the plurality of data sets by a weight indicative of the representativeness of the member associated with the each set, wherein a weight aᵢ for a data set obtained from a member i of the population is calculated as:

$${a}_{i} = \frac{{n}_{i}^{p}}{{n}_{i}^{s}} ,$$

where nᵢ^{p} is the number of members in the population who share a same set of characteristics with the member i, and nᵢ^{s} is the number of members associated with the collected data who share the set of characteristics.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures illustrating example embodiments of the present invention.

FIG. 1 schematically illustrates formation of a risk prediction model in manners exemplary of the present invention;

FIG. 2 is a flowchart illustrating exemplary steps performed at a computing device of FIG. 1;

FIGS. 3 to 5 and 7 are flowchart further illustrating steps of FIG. 2;

FIG. 6 is a block diagram illustrating division of data as performed in a step of FIG. 5;

FIG. 7 is a block diagram illustrating sub-steps of yet another step in FIG. 2; and

FIG. 8 illustrates two exemplary disease risk curves determined in manners exemplary of the present invention.

### DETAILED DESCRIPTION

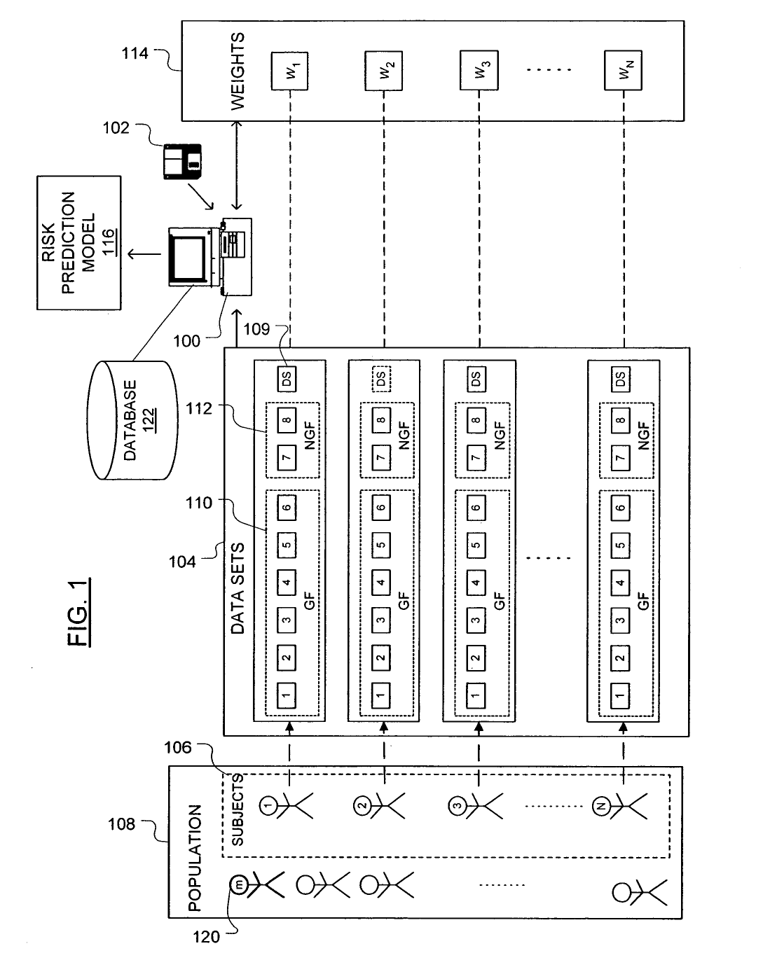

FIG. 1 graphically illustrates formation of a risk prediction model 116, in manners exemplary of embodiments of the present invention. Example risk prediction model 116 is formed to predict the likelihood that a particular patient 120 that is a member of a population 108 will develop a particular disease of interest. As will become apparent, risk prediction model 116 may be effective in predicting risk of a number of patients within population 108.

As illustrated, example risk prediction model is determined, using a general purpose computing device 100, executing software exemplary of embodiments of the present invention. As such, computing device 100 includes a processor and processor readable memory, for storing processor executable instructions adapting computing device 100 to function in manners exemplary of embodiments of the present invention. The memory may be any suitable combination of dynamic and persistent storage memory, and may therefore include random-access memory; read-only memory; and disk memory.

As illustrated, a database 122 is also preferably hosted on (or in communication with) computing device 100. Database 122 may, for example, be any suitable relational; object oriented; or other database. A suitable database engine for querying; storing; updating; and deleting records within the database is also preferably stored for execution at computing device 100.

Optionally, computing device 100 may further include peripherals such as keyboard; display; printer; speakers, and the like. Optionally, computing device 100 may also include a network interface interconnected with a data network, such a local or wide area network, or the public internet. Suitable software to use these peripherals may also be stored at device 100, for execution as required.

Software performing steps exemplary of the present invention may be loaded into memory of computing device 100 from a computer readable medium 102. Computer readable medium 102 can be any available medium accessible by a computer, either removable or non-removable, either volatile or non-volatile. Such computer readable medium may comprise random-access memory (RAM) or read-only memory (ROM), or both. A RAM may be dynamic (DRAM) or static (SRAM). A ROM may include programmable ROM (PROM), erasable PROM (EPROM), and electrically erasable PROM (EEPROM) such as Flash Memory. By way of example, and not limitation, computer readable media include memory chip, memory card, magnetic cassette tape, magnetic cartridge, magnetic disk (such as hard disk and floppy disk, etc.), optical disc (such as CD-ROM, CD-R, CD-RW, DVD-ROM, DVD-R, and DVD-RW).

Exemplary of the present invention, a plurality of data sets 104 used in the formation of prediction model 116 are collected from subjects 106 within in population 108. Preferably, these are stored within database 122. That is, for any particular disease of interest, software exemplary of the present invention may be used to store, collect and process data entries that are indicative of a risk of that disease. Each data entry corresponds to an observed indicator of the disease risk for the disease of interest, as observed in a sampled subject. For any particular disease of interest, multiple indicators may be pre-defined by a knowledgeable user of computing device 100. As will become apparent, the choice of indicators that are sampled and for which data entries are stored for analysis at computing device 100 depends largely on the nature of the disease of interest.

For a disease of interest one data set 104 is collected from each sampled subject 106. Each data set 104 includes an indicator 109 indicating the disease status (DS) of the corresponding subject. Indicator 109 reflects whether or not the sampled subject has been diagnosed with the disease of interest at the time the associated data set 104 is collected. Indicator 109 may have two possible values, e.g. “0” for a healthy subject and “1” for sick subject. Each data set 104 also includes a plurality of data entries 110 and 112, reflective of indicators of risk factors, as sampled from an associated subject.

In the illustrated example, each of data sets 104 includes eight data entries, one for each of eight indicators of risk, of which six (corresponding to entries 1-6) represent indicators of genetic risk factors (GF) and two (indicators 7 and 8) represent indicators of non-genetic risk factors (NGF). For clarity, data entries representative of indicators of genetic risk factors are collectively referred to herein as genetic data 110 and data representative of non-genetic risk factors are collectively referred to herein as non-genetic data 112.

For example, typical genetic indicators of disease risks include the presence or absence of genetic markers such as SNPs and other polymorphisms in a subject. These genetic markers are segments of a DNA sequence with an identifiable physical location that can be easily tracked and used for constructing a chromosome map that shows the positions of known genes, or other markers, relative to each other. As is conventional, a genetic code is specified by four nucleotide “letters” A (adenine), C (cytosine), T (thymine), and G (guanine). SNP variations occur when a single nucleotide, such as an A, replaces one of the other three nucleotide letters—C, G, or T. An example of an SNP is the alteration of the DNA segment GGMTTA to GTMTTA, where the second “G” in the first snippet is replaced with a “T”. The latter segment GTAATTA may serve as a genetic marker.

SNPs that occur in protein coding regions give rise to variant or defective proteins. Even SNPs outside of “coding sequences” may result in defective protein expression, though much less likely. Defective protein expressions are potential causes of genetic diseases. Thus, some SNPs may predispose a person to diseases, may confer susceptibility or resistance to a disease, and may determine the severity or progression of disease. In addition, whereas many SNPs do not produce physical changes in people and it has never been documented that a single SNP actually causes a complex disease, SNPs may serve as genetic markers of diseases because they are usually located near genes associated with a certain disease. Thus, the presence of a genetic marker in a subject's DNA indicates the presence of a gene associated with the disease. Further, the collective effect of multiple SNPs and other types of genetic polymorphisms are believed to affect the risk of a complex disease.

Data indicative of a genetic factor may be represented by data entries 110 with corresponding integer values, such as zero, one, and two. For instance, where the genetic marker is a particular allele at a given locus, the value of zero may indicate the absence of the genetic marker in the associated subject 106; the value of one may indicate the presence of the genetic marker in the heterozygous form; and the value of two may indicate the presence of the genetic marker in the homozygous form. Genetic factors may also be represented in other formats and may have more than three values.

The term non-genetic factor is used in a broad sense herein. Non-genetic factors may include any environmental factors that may affect the development of the disease, such as age, weight, height, lifestyles such as smoking status and diet, living conditions, education, medical history of certain diseases, and the like. Non-genetic factors may also include factors that have a genetic origin but are not being genotyped, such as sex, race and family history of the disease to be predicted. As will become apparent, age should be included if the risk prediction model includes time as a variable, such as in the case of survival models (see details below). Non-genetic genetic data 112 for any subject 106 may accordingly include a combination of numerical (including binary) values reflecting the identified non-genetic factors.

Conventionally, to be able to predict a disease risk, the disease is correlated with the presence or absence of relevant genetic markers and environmental factors. This is done, in part, by comparing the genotypes of sick individuals (often referred to as “case” subjects) with genotypes of healthy individuals (often referred to as “control” subjects). If a SNP, or a combination of SNPs, appears more frequently in the case subjects than in the control subjects, then such a SNP or combination is considered a possible “marker” of the particular disease. The comparison is typically made by fitting all of available sample data to a statistical model. For a single marker, the strength of the marker depends on the disparity in frequencies and the reliability of the marker depends directly on how accurately the subjects (also referred to as samples) represent the general population.

When there are multiple markers, the analysis may become very involved and the computation intensifies as the number of risk factors increases. Analysis is particularly difficult when there is a large number of both genetic and non-genetic factors that need to be taken into account. Yet, most diseases are associated with multiple risk factors. For example, many chronic, non-infectious diseases such as cancers, coronary artery disease, diabetes, asthma, schizophrenia and Alzheimer's disease have complex modes of inheritance. They are commonly known as “complex diseases”. Unlike simple genetic diseases such as thalassemia and hemophilia for which a gene mutation is the only cause of the disease, complex disorders are a product of the interactions between multiple genes and environmental factors. The genetic factors that contribute to an individual's susceptibility to complex diseases are usually found on many different genes. Most of these are SNPs but each of them does not constitute a causal mutation. It has been postulated that particular combinations of SNPs may render an individual susceptible to a particular complex disease. Cargil et al., “Characterization of single-nucleotide polymorphisms in coding regions of human genes,” Nature Genetics 22:231-8 (1999).

Many existing methods for selecting significant predicting factors of disease risks simply exclude certain genetic markers if these markers co-exist with strong environmental factors.

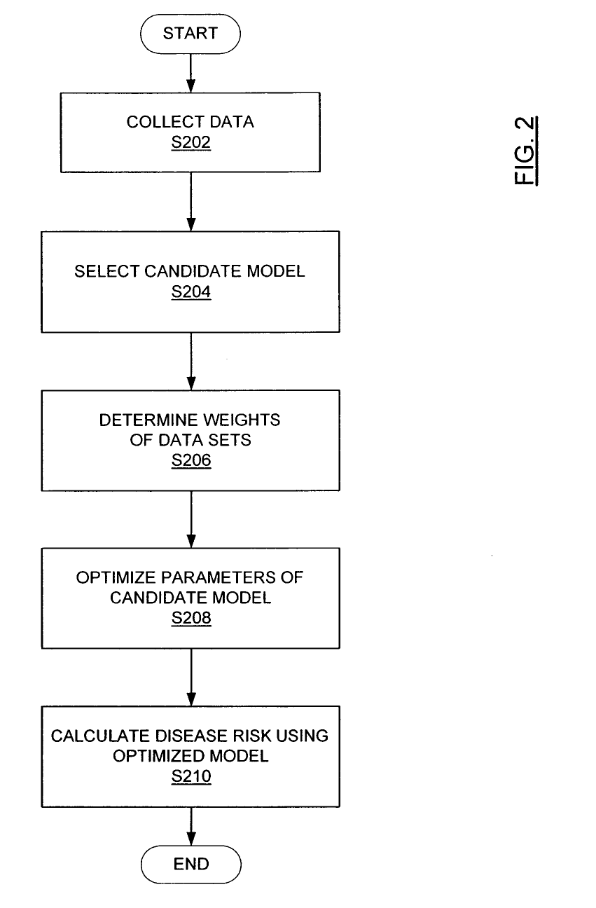

Steps performed by computing device 100 in manners exemplary of embodiments of the present invention are illustrated in overview in FIG. 2. As will become apparent, data sets 104 (FIG. 1) from a plurality of subjects 106 are collected in step S202. A candidate statistical model for calculating the disease risk is selected at step S204. The candidate model is explicitly dependent on non-genetic factors only and has a plurality of parameters. The number of parameters may be equal to or less than the number of non-genetic factors analyzed. Where the candidate model is expressed as a mathematical equation, such as the risk function of a Cox model, the parameters may be expressed in the form of coefficients of the equation, each coefficient associated with a non-genetic factor. The candidate model can be selected from a plurality of candidate models stored at computing device 100, which may comprise models other than a Cox model. In step S206, a corresponding weight 114, used in assigning a statistical significance to collected data, is calculated for each data set 104 with reference to a particular genetic make-up, as will be further described below. The weights for data sets that have like genetic data are the same. In step S208, the parameters of the candidate model, such as the coefficients of the Cox model, are optimized by fitting data sets 104 to the candidate model, taking into account of weights 114. The resulting model may be taken as the risk prediction model 116. In step S210, a disease risk for a subject of interest is calculated using the risk prediction model 116.

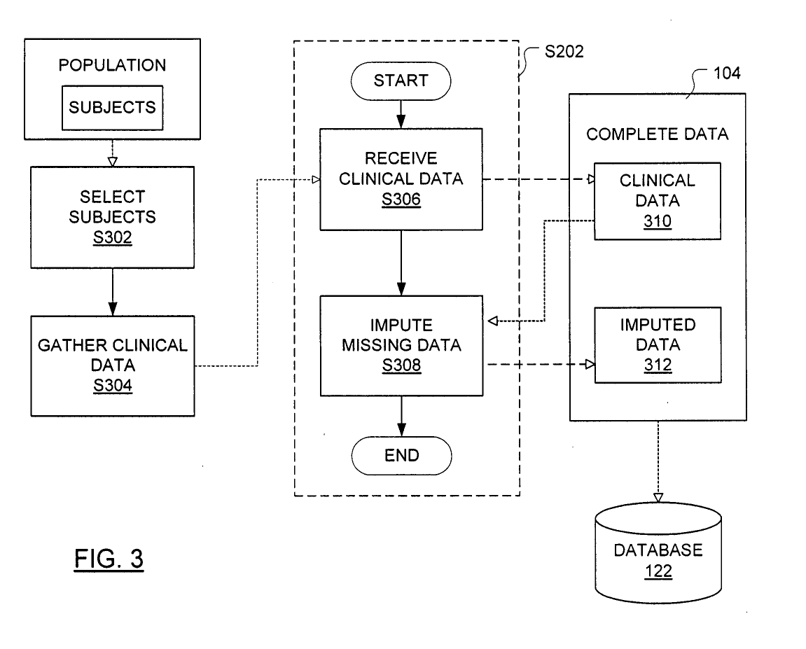

Collection of data from subjects 106, as performed by computing device 100 in step S202, is more particularly illustrated in FIG. 3.

Prior to the performance of step S202 by computing device 100, subjects initially are selected manually from population 108 in step S302. Once a subject is selected, data is extracted from the subject in step S304. The selection of subjects 106, as performed in step S302 is known in the art as “sampling,” may be carried out by any number of ways understood by a skilled person in the art. For instance, subjects 106 may include patients attending or admitted to certain medical clinics, hospitals, and other institutions for treatment of the disease in question, as well healthy individuals attending to these clinics, hospitals and institutions for medical check-ups and other purposes. Another exemplary way of sampling is to randomly survey the population in a given geographical area by, e,g., questionnaires, telephone calls, in-person interviews and etc. Subjects 106 may be selected and accumulated over a period of time, over different geographical locations, from different risk studies including past studies, or from existing databases. Subjects 106 may also be selected in a variety of different manners.

In practice, it may be difficult to select subjects truly representative of the population. It is often too expensive and even impractical to do so. The selection is often not truly random. The number of samples may not be large enough. Advantageously, subjects 106 need not be truly representative of the population 108 because over- or under-representation of the true population 108 can be compensated as described herein. Nonetheless, it is preferable that the subjects 106 represent the population 108 well. For example, it is preferable that the sample size is sufficiently large and the subjects are reasonably randomly chosen. Further, the data sets 104 obtained from all subjects 106 should collectively contain sufficient information about the particular disease of interest.

Particularly, subjects 106 should include sufficient numbers of sick and healthy subjects. Sick subjects are those who have been clinically diagnosed with the disease in question at the time of sampling and healthy subjects are those who have not have been diagnosed with the disease at the time of sampling. Advantageously, sampled subjects need not be classified as cases and controls in the conventional sense. In conventional case-control type studies, the status of a subject as being a case or control does not change over time, and therefore some subjects are inevitably misclassified. Specifically, a subset of the control group would inevitably develop the disease given time. Such misclassification adversely affects the result of any analysis that relies on the classification. Unlike in conventional disease risk studies, disease risks may be treated as functions of time (i.e. age) and healthy subjects are simply those who have yet to develop the disease but eventually may if given time.

Thus, for each sampled subject 106, a data set 104, including genetic data 110 and non-genetic data 112, and an indicator of illness 109 is extracted. This is provided to computing device 100 in step S306, and stored in database 122, as clinical data 310. The stored data may be gathered using any existing clinical techniques. For example, in order to obtain genetic data 104, blood samples may typically be taken from the subjects. Genomic DNA may be prepared from the blood samples, for example, according the method described in Parzer et al. “A rapid method for the isolation of genomic DNA from citrated whole blood,” Biochemical Journal 273: 229-231 (1991) (“Parzer”). Simultaneous genotyping can be carried out by for example an arrayed primer extension as described by Syvanen et al. “A primer-guided nucleotide incorporation assay in the genotyping of apolipoprotein E,” Genomics 8:684-92 (1990) (“Syvanen”). Other known techniques for genotyping may also be used. Non-genetic information may be obtained through questionnaire, interview, observation, and other clinical examining techniques such as measurement of heartbeat or blood pressure, radio-active or electromagnetic scan, chemical or biochemical analysis of body fluid or tissue samples, and the like.

Often, not all desired data from all subjects 106 can be clinically obtained. Some data is usually missing. For example, a particular subject may have forgotten to answer a question on a questionnaire concerning family medical history or simply did not know; certain DNA analysis may have failed to yield results, and thus it is not known whether certain genetic markers are present. A common practice in conventional disease risk studies is to discard samples that have incomplete information, even if only one piece of information on a subject is missing. This practice reduces, often drastically, the sample size. It may also adversely affect the sample's representation of the population by excluding certain subgroups of the population. For example, it may be that most subjects with missing data have lower income or had died because of the disease.

To avoid wasting clinical data, any missing data may be optionally imputed by computing device 100 in step S308 based on data actually obtained (clinical data 310). Imputation may also be performed based on a combination of clinical data 310 and previously imputed data. Imputed data 312 is also stored in database 122.

Imputation techniques are generally known. Exemplary conventional imputation techniques are multiple imputation techniques described in Schaefer, Analysis Of Incomplete Multivariate Data, London: Chapman and Hall (1997) and Rubin, Multiple Imputation for Nonresponse in Surveys, New York: John Wiley & Sons (1987). In essence, multiple imputation has three phases. First, the missing data is filled multiple times to generate multiple complete data sets. Data used to fill missing data may be generated in various manners, such as completely randomly, or drawn either evenly or randomly from a given distribution. For a monotone missing data pattern, a parametric regression method may be appropriate. For an arbitrary missing data pattern, the Markov chain Monte Carlo (MCMC) method, which creates imputation data by using simulations from a Bayesian prediction distribution for normal data, may be used. To reduce computation, missing values may be calculated for one variable or a subset of all variables at a time, as described in more detail below. Next, the multiple complete data sets are analyzed according to standard statistical analysis procedures. Thereafter, the results from the multiple complete data sets are integrated or combined into one complete set, having imputed values in place of missing data, which can be used for subsequent analysis.

The choice of imputation technique may depend on the sample size, missing data pattern, and the number and types of indicators of risk factors. When the number of indicators is small, existing imputation techniques such as the conventional multiple imputation technique may be adequate. However, the amount of calculations required increases quickly with increasing number of indicators. When the number of indicators is large, existing imputation techniques would require extensive computation resources, even more than is practically available. As mentioned, there are usually a large number of genetic markers associated with a complex disease.

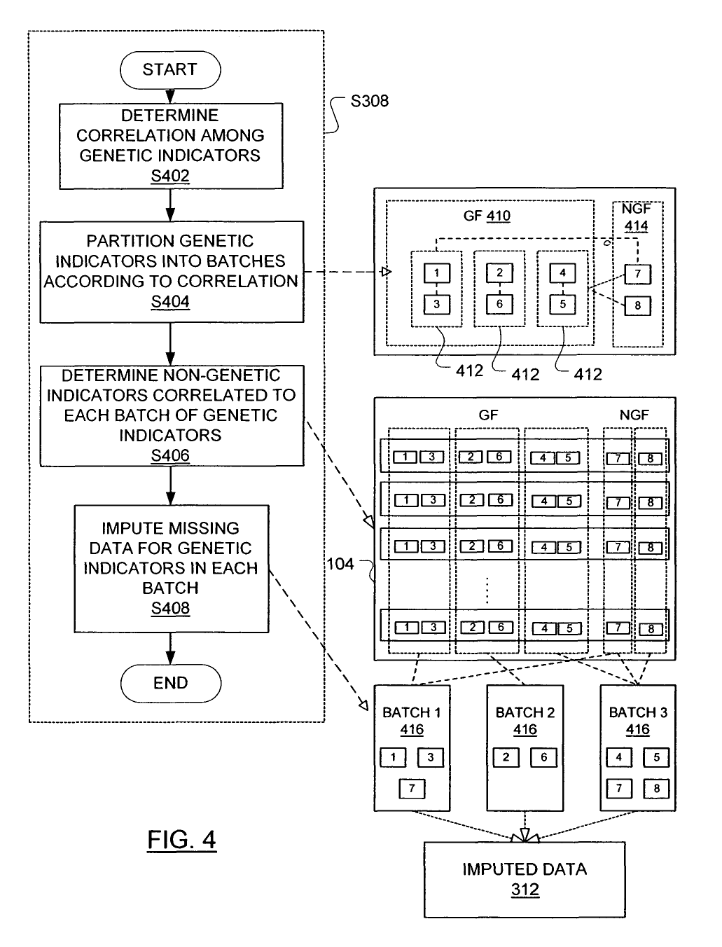

Thus, an exemplary embodiment of the present invention provides an imputation method that reduces the calculations required for imputing missing genetic data, as illustrated in FIG. 4. In this method, instead of imputing missing data for all genetic indicators at once, missing genetic data is imputed separately in batches of genetic indicators, one at a time. As the number of indicators in each batch is small, the amount of computation is significantly reduced. Since data of correlated indicators is likely to influence each other, it is preferable to have correlated indicators grouped together. When the number of non-genetic factors is small, as it is usually the case, missing values of non-genetic indicators can be inputed at once without significant computation difficulty.

Therefore, in step S402, the correlation among the genetic indicators 410 is determined. Typically, this can be done by calculating the correlation matrix for the genetic indicators 410 from the genetic data 110 using conventional statistical analysis techniques. Other statistical methods for determining correlation between data may also be used. For example, the dependence between two factors may be assessed by a chi-square test using a table formed by cross-tabulating data for the two factors. It is possible that the correlation among the genetic indicators 410 be determined based on previously obtained data or only part of the genetic data 110. In the example illustrated in FIG. 4, continuing from the example data sets illustrated in FIG. 1, indicators 1 and 3 are correlated, so are indicators 2 and 6, and indicators 4 and 5.

In step S404, the genetic indicators 410 are partitioned according to their correlation with each other. Strongly correlated genetic indicators are grouped together into one batch 412. There may be one or more batches 412 of strongly correlated genetic indicators. In the example illustrated in FIG. 4, there are three batches 412 of correlated genetic indicators 410: indicators 1 and 2 in batch one, indicators 2 and 6 in batch two, and indicators 4 and 5 in batch three.

Steps S402 and S404 may be carried out using any existing statistical classification or structure-detecting methodology, such as factor analysis, principled components analysis, correspondence analysis, other techniques in data mining, and the like. The total number of batches 412 and the number of indicators 410 in each batch 412 may be adjusted by requiring a stronger or lesser correlation between indicators 410 within each batch 412.

In step S406, non-genetic indicators 414 that are correlated to each batch 412 of genetic indicators are determined. Continuing from the above example, it may be determined that indicator 7 is correlated to batch one, indicators 7 and 8 are correlated to batch three, and none of the non-genetic indicators is correlated to batch two.

In step S408, conventional imputation techniques may be applied to impute missing data for each batch 412 of genetic indicators 410 separately. For each batch of genetic indicators, only genetic data for the indicators within the batch and non-genetic data for non-genetic indicators that are correlated to the batch (as determined in step S406) are used in imputing data for the indicators in that batch. Again continuing the above example, imputation of missing data in data sets 104 may be carried out in three batches 416. In batch 1, the missing data for indicators 1 and 3 are imputed together with all clinically collected data for indicators 1, 3 and 7; in batch 2, the missing data for indicators 2 and 6 are imputed together with clinical data for the two indicators; and in batch 3, the missing data for indicators 4 and 5 are imputed with clinical data of indicators 4, 5 ,7 and 8.

Since the number of indicators in each batch is less than the total number of indicators, the imputation calculations required for each batch is much less than that for imputing all missing data at once. The calculations for all groups combined are still significantly less than calculations required for imputation all missing data at once. For example, comparing with imputing data for one thirty-indicator group, imputing data for six five-indicator groups could reduce computation by a factor of more than 10¹⁰. Thus, even for a large number of genetic markers, imputation of missing data with the procedure described above is feasible with currently available computing resources.

Clinical data 308 and imputed data 310 together form the complete data sets 104. The complete data sets 104 may be stored in a database 312 within memory of device 100. During later analysis, clinical data 308 and imputed data 310 will not be distinguished. It can be appreciated that by imputing missing data, no clinical data need to be discarded. Useful information need not be wasted. The sample size may be maintained. Distortion to the representativeness of the sample due to discarding data may be avoided.

As should be appreciated, once suitable data sets have been acquired, gathering data from subjects and imputing data may no longer be necessary. Thus, step S306 and S308 could be replaced by simply accessing a database (not necessarily database 122 of FIG. 1) that stores sufficient data to allow prediction of disease risk.

To analyze the collected data, a candidate statistical model is selected (S204). The candidate model can be any suitable survival model, such as a Cox model. In an exemplary embodiment of the invention, a Cox model is used. To reduce the number of variables involved in the fitting, the hazard function is assumed to depend on non-genetic factors only. That is, xᵢ are all indicators of non-genetic factors.

However, genetic data are not simply discarded. Genetic data are used in assigning statistical significance to data sets. Intuitively, it may be expected that not all of the data sets have the same statistical significance. To determine a risk prediction model for a given combination of genetic data, all data sets having the same combination of genetic data are likely most significant as the base hazard function may be the same for people having the same genetic make-up. However, data sets associated with members having different genetic make-up may have less statistical significance.

To take into account of the effects of genetic factors, two assumptions may be intuitively made: first, the optimal models for subjects with different combinations of genetic indicators could be different; second, data sets with like genetic data would have the same statistical significance and data sets with unlike genetic data would have different statistical significances. The statistical significance of a data set is indicated by a corresponding data weight 114. A data set may have different statistical significance with reference to different combinations of genetic indicators. Thus, a corresponding weight is determined with reference to a particular combination of genetic indicators. As can be appreciated, use of weights allow the generation of a conditional probability model for a given combination of genetic indicators. The corresponding weights 114 reflect the effects of genetic risk factors on the disease risk and the interplay between different risk factors, particularly between genetic and non-genetic risk factors.

The corresponding weights 114 for all data sets 104 can be determined as described below. While the corresponding weights may be separately determined for each data set 104, such an approach may require extensive computation if the number of data sets is large or the number of genetic indicators is large.

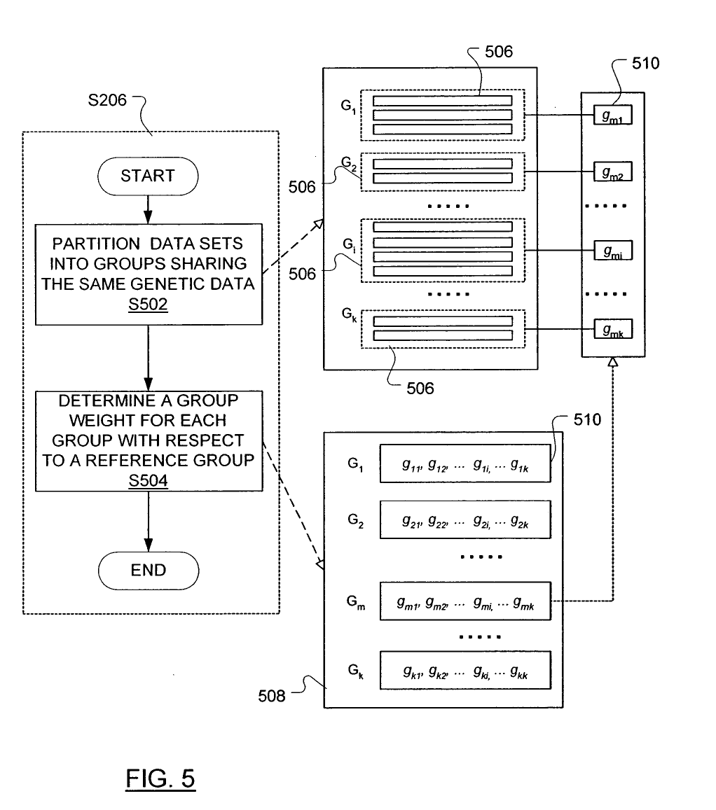

As weights are calculated with reference to a particular combination of genetic factors, data sets 104 having identical values of genetic factors are assumed to have the same statistical significance. As such, to reduce calculations required, corresponding weights 114 are preferably determined in groups as illustrated in FIG. 5. Specifically, in step S502, the collected data sets 104 are divided into a plurality of groups 506 (G₁, G₂, . . . Gᵢ . . . Gₖ). These groups are formed so that the genetic data 110 of the data sets 104 in each group 506 share some common features.

The data sets 104 may be partitioned in any number of ways to form groups 506. For example, one possible division forms groups 506 each having identical genetic data 110. For instance, assume that each genetic marker has three possible values, 0, 1, and 2, indicating whether 0, 1 or 2 copies of a particular genetic marker is present in a subject. The genetic data in each data set consists of a series of 0s, 1s, and 2s. The criterion for division may be that each data set in the group must have an identical sequence of 0s, 1s and 2s, i.e., all sets in the group have a set of identical markers, which indicates that all subjects associated with this group have the same combination of genetic markers. When the number of genetic markers is large, this exemplary approach often results in too many groups with many groups having no data.

An alternative division might require only genetic data 110 corresponding to some selected genetic indicators in each data set of a group to be the same. For example, the exemplary data sets 104 of FIG. 1 could be divided into 9 groups 506 (k=9) according to the values of only two indicators. This way, the number of groups 506 (k) may be limited and it can be ensured that there are a certain number of data sets in each group 506.

Even if a given indicator is used as a criterion for division, the values of the indicator in each group Gᵢ need not be identical. In appropriate cases, it may be appropriate to only require that the values are of a certain type or within a certain range. For example, values may fall within two ranges, low, and high, and may accordingly be grouped into two different groups 506.

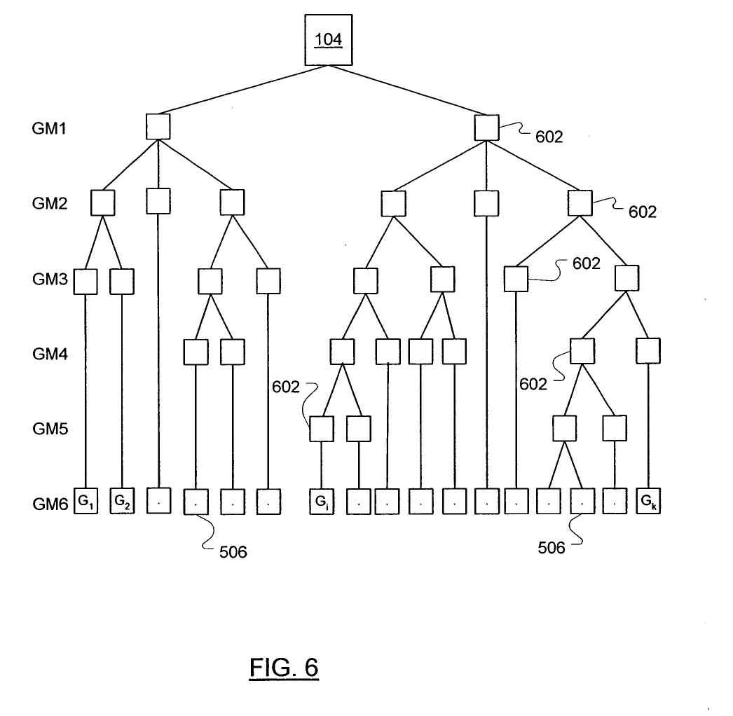

FIG. 6 illustrates an exemplary procedure for partitioning data sets 104 using the genetic markers. The results of partitioning can be represented by a tree 600. In the example illustrated in FIG. 6, six genetic markers (GM1-GM6) are used to split the data sets 104 into k groups 506.

At the top level, data sets 104 are divided into two intermediate groups 602 depending on their values of GM1. For example, all data sets in which GM1=1 may be grouped together on the left hand side, while all data sets in which GM1=0 are grouped together on the right hand side. At the next level, the division is made depending on the values of GM2. Since GM2 have three possible values (0, 1, and 2), each intermediate group is further divided into three intermediate groups. Similarly, the new intermediate groups are further split using GM3. However, it may be decided (as described below) that some intermediate groups 602 (e.g., the middle group on both sides after the GM2 split) need not be further divided according values of GM3. These undivided groups then become terminal groups 506. The process continues until the data sets have been divided using all six genetic markers, resulting in the terminal groups 506 (G₁, G₂, . . . Gᵢ . . . Gₖ). Thus, each level of tree 600 represents a particular grouping or partition of the data sets 104. Each node 602 represents an intermediate group. At each level, the intermediate groups 602 may be split with respect to one genetic marker (GM). The terminal nodes 506 represent the final grouping of the data sets 104.

As noted, not all intermediate groups 602 at all levels are necessarily split. Whether to split a particular intermediate group 602 may be determined based on various criteria such as the numbers of data sets in the resulting groups, any improvement in the ultimate fitting results, and any other appropriate considerations.

To ensure that each group 506 has sufficient data sets for proper statistical analysis, the criteria for splitting may include a minimum size requirement, i.e., the resulting groups must have more than a minimum number of data sets.

To reduce the number of resulting groups 506, it may be required that a node 602 is only split if the splitting will improve the quality of later data analysis. For instance, if a later fitting result depends on the partition of the data sets, the criteria for splitting may include a goodness of fit for the fitting. Any goodness of fit criterion for the fitting, such as deviance, may be used. The goodness of fit may be evaluated based on a pre-set absolute limit, or a relative comparison between fitting results with and without the splitting; a node 602 needs not be split if the goodness of fit with splitting does not improve over the result without splitting. The criteria for splitting may also include a likelihood ratio test. If the test yields a likelihood ratio higher than a pre-defined value (such as 5%) then the node may be split. Otherwise, the node is not split.

In any event, a particular split will be ultimately carried out only if the criterion for splitting is satisfied to ensure that the number of groups is manageable, the partition is statistically supported by the data, and the partition may be reliably used in further analysis.

To that end, a tree-pruning procedure may be optionally additionally carried out either during or at the end of partitioning step S502, in which two terminal groups 506 from a split are re-combined if the likelihood ratio for their splitting is too small according to a standard likelihood ratio test.

As can be appreciated, an intermediate group 602 may be further divided at a lower level even though it was not split at a higher level. Further, the order of genetic markers used may affect the ultimate grouping. The order may be chosen randomly or based on certain criterion. For example, different orders may be tested to select the one that produces the best result.

While the procedure described above and illustrated in FIG. 6 is fast and facilitates incorporation of the corresponding weights 114 in the calculation (as will become clear from the description below), the partitioning of data sets 104 may be carried out using other existing classification techniques. Different partitioning methods may result in different numbers of groups 506, or different constructions of the same number of groups 506. An advantage of partitioning data sets 104 is that it facilitates the analysis of effects of both genetic and non-genetic risk factors without incurring impractical expenditure of computing resources, the ultimate choice of grouping may be made based on a balanced consideration to increase the ultimate quality of the data analysis and to reduce the amount of calculations required, as exemplified in the example embodiments described above.

Whatever the criteria, each data set 104 is included in one and only one group 506. Each member of the population 108, particularly the member for whom a risk of disease is to be assessed, also belongs to one group 506.

Once the sets of data are partitioned into groups, a group weight 510 (denoted by gₘᵢ) is determined for a group Gᵢ 506 with respect to a reference group 508 Gₘ, These weights are used as weights 114 for all data sets 104 in the group Gᵢ. The first subscript in the group weight symbol (e.g. the “m” in gₘᵢ) indicates the reference group and the second subscript (e.g. “i”) indicates the group for which the group weight is to be determined; e.g. g₁₂ is the group weight for group 2 with respect to reference group 1. k group weights 510 are determined with respect to each reference group 508.

Since there are k possible reference groups for any one of k groups, a total of kxk group weights 510 may be determined. It is not always necessary but may be convenient and efficient in some situations to determine and store all kxk group weights 510 in advance for later risk calculations. The reference group 508 may be chosen according to the genetic data of a particular member (“m”) of the population 108 for whom a risk prediction calculation is to be carried out. That is, if the particular member would have belonged to group Gₘ, had the member been a subject 106, group Gₘ is then the reference group 508 with respect to which the group weights 510 will be determined.

Group weights 510 may be determined in various manners to properly account for the effects of variation in genetic data on the disease risk. Generally, if the genetic data 110 in a group 506 is similar to that in the reference group 508, the group 506 should have a high group weight 510. If the genetic data 110 is dissimilar, the group 506 should have a low group weight 510. The closer the similarity, the higher the group weight 510. The similarity and closeness may be evaluated based on numerical values or classifications or both. The group weights 510 may be determined based on previously acquired knowledge or based on analysis of the data in the data sets 104, or a combination of both.

For example, in an exemplary embodiment of the present invention, the reference group Gᵢ 508 is assigned a group weight 510 of one, i.e., gᵢᵢ ≡1, whereas all other group weights 510 (gᵢⱼ, where i≠j) have values between zero and one. The other group weights 510 are optimized simultaneously by minimizing the sum of squared residuals for the data in Gᵢ, as follows. For a given set of group weights, optimize the model parameters (such as coefficients βᵢ) by fitting non-genetic data in all data sets except those in group Gᵢ to the candidate model, with each data set being weighted by the corresponding weight 114. The optimized model is then used to calculate the total residual of all data in group Gᵢ. The set of group weights that produces the minimal total residual for group Gᵢ is the optimal set of group weights.

As can be appreciated, the residuals can be any suitable ones. For example, deviance residuals may be used and the target residual function may be a residual sum of squares (RSS). Further, the minimization procedure may utilize a standard multi-fold cross-validation method to increase the reliability of the optimization.

Because the calculation of group weights 510 in this manner is computationally intensive, it may be desirable to carry out these calculations in a distributed or parallel manner as described further below.

The corresponding weight 114 of each data set 104 within a group 506 equals the group weight 510 for that group 506. Thus, the corresponding weights 114 (wᵢ) for all data sets 104 can been determined once a reference group 508 has been chosen and the group weights 510 for that reference group 508 have been determined.

Optionally, each weight 114 (wᵢ) may be adjusted to reflect the subject's representation in the population 108 and thereby compensate for deficiencies in sampling. If the subjects are randomly chosen and are truly representative of the population, no adjustment is necessary. Otherwise, some weights may be adjusted differently than others. For example, if a sub-population of the population 108 is over-represented in the sampled subjects 106, any data set 104 obtained from a subject 106 of that sub-population is adjusted by a low adjustment factor. Similarly, a data set 104 from a subject 106 of an under-represented sub-population adjusted by a high adjustment factor. For example, the adjustment factor aᵢ, for a weight corresponding to a data set i, may be calculated as follows:

$${a}_{i} = \frac{{n}_{i}^{p}}{{n}_{i}^{s}} ,$$

where nᵢ^{p} is the number of members of the population 108 who share a set of characteristics with the subject i, and nᵢ^{s} is the number of subjects 106 who share the set of characteristics. The set of characteristics may include any characteristic that may be shared by more than two individuals. For example, the characteristics may include age, sex, nationality, ethnic background, health history and others. The set of characteristics may vary depending on available data, such as demographic and health statistical data for the population 108. The set of characteristics may include one or more of the indicators of risk factors. Imputed data may also be used for calculating the adjustment factor.

Once an adjustment factor aᵢ is calculated, the corresponding group weight wᵢ, may be replaced by aᵢ·wᵢ (i.e. scaled by aᵢ). Adjusted weights wᵢ may be used in the data analysis to compensate the adverse effects of imperfect sampling, thus allowing good results to be obtained even though the subjects 106 are not truly representative. As a result subjects 106 need not be truly randomly selected and the sample size need not be very large reducing the cost of sampling or increase the reliability of results obtained from imperfect samples.

Adjustment factors aᵢ may be alternatively calculated before imputing missing data. It is also possible to use adjustment factors aᵢ in the imputing process (step S306). Further, adjustment factors may be recalculated after imputation.

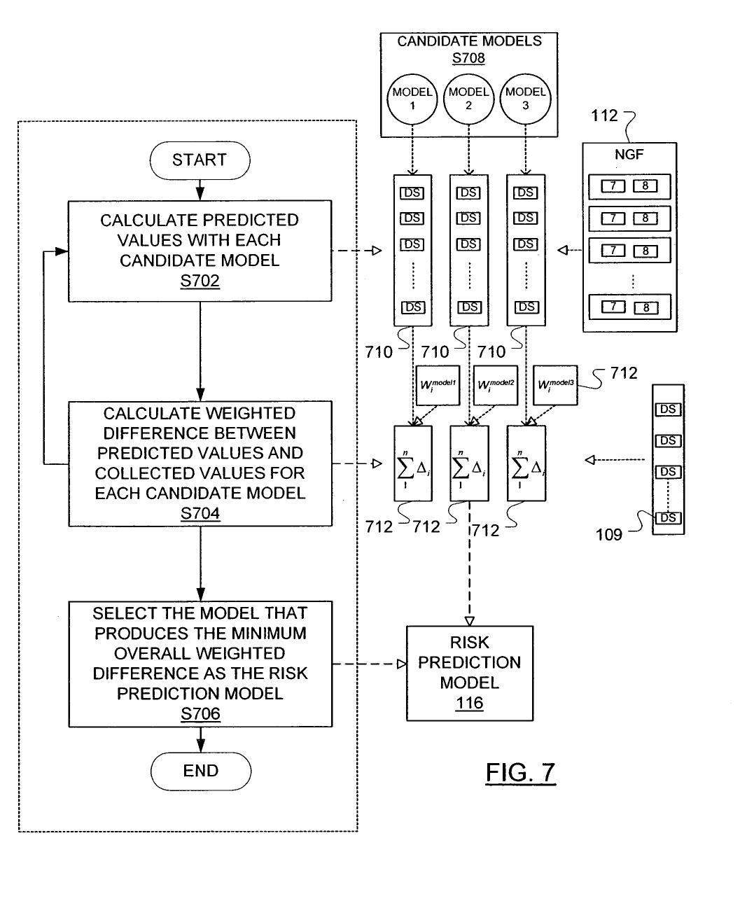

Once weights wᵢ are calculated, an optimal candidate model 116 may be determined for a given combination of genetic data, by fitting non-genetic data 112 in all data sets 104 to the candidate model and optimizing the parameters (such as coefficients βᵢ). The goodness of fit is assessed taking into account of the corresponding weights 114. Specifically, errors attributable to samples having low weights are thus less significant than those of samples having high weights. Optionally, several candidate models may be stored at computing device 100 and used to find an optimal statistical model for patient 120. So, steps 700 illustrated in FIG. 7 may be performed as part of steps S204 to S208 in FIG.2. As illustrated, a statistical model for calculating a disease risk of a patient belonging to a reference group 508 is determined by statistically analyzing only the collected non-genetic data 112. Each data set 104 is given a statistical significance corresponding to its corresponding weight 114. The corresponding weights 114 for each candidate model may be different and are calculated using the respective models. In steps S702, S704 and S706, the non-genetic data 112 may be fit to one or more candidate models 708, with one of the fitting criterion dependent on the corresponding weights 114. For each model, different weights wᵢ are calculated, as described above with reference to step S206. A model may be any statistical tool for representing relationships between different variables, such as functions, tables, matrices, graphs, and the like. A model may also include combinations of these tools. Specifically, a model may be a function or a family of functions. Particularly, Cox models with different number of parameters may be used. For example, the hazard function may depend on different combinations of non-genetic factors and have different number of coefficients. The model that produces the minimal total residual may depend on all of the non-genetic factors for which data are collected or it may depend on only a subset of the non-genetic factors included in data set 104.

For example, candidate models 708 may be functions of non-genetic indicators 112 that can be used to predict the disease status (DS) of subjects 106 in the reference group 508. That is, given the values of non-genetic indicators of a subject 106 in the reference group 508, each of the candidate function can be used to calculate a value of DS 710 for the subject 106. A fitting criterion may be the sum of weighted deviates 712

$$( {\sum }_{1}^{n} {Δ}_{i} )$$

of the fits. A deviate is the difference between an observed value and the corresponding predicated value. In the example illustrated, the observed values are the values of disease status indicator 109 in the collected data sets 104 and the predicted values are the values of indicator 710 calculated using the candidate models 708. A weighted deviate (Δᵢ) is a function of the deviate and the corresponding weight 114 of the relevant data set 104. For example, the weighted deviate may be a product of the deviate and the corresponding weight 114, i.e., Δᵢ=wᵢ|DS_{predicted}−DS_{observed}|=wᵢ|rᵢ|. The weighted deviate may be otherwise calculated in accordance with known practices of statistical analysis. For example, define Δᵢ=wᵢ(DS_{predicted}−DS_{observed})²=wᵢrᵢ², or more generally, Δᵢ=wᵢφ(rᵢ). Then, the fitting criterion will be to minimize the value of

$${\sum }_{1}^{n} {w}_{i} \vert {r}_{i} \vert , or {\sum }_{1}^{n} {w}_{i} {r}_{i}^{2} ,$$

or

$${\sum }_{1}^{n} {w}_{i} ϕ ( {r}_{i} ) ,$$

respectively. As will be understood by a person skilled in the art, the weighted sum of deviates 712 can be a measure of the goodness of fit and can be used for comparing results from different fits. For example, the model that produces the minimum value of the weighted sum of deviates 712 is typically considered the best model and may be selected as the risk prediction model 116 for the particular reference group 508. In the example illustrated in FIG. 7, candidate model 2 produces the lowest weighted sum of deviate among the three candidate models 708. That is, it fits data sets 104 best using the described techniques and is therefore used as the risk prediction model 116. As can be appreciated, the corresponding weight 114 of a data set 104 determines the statistical significance of the data set 104

In an exemplary embodiment, the goodness of fit is assessed by calculating the deviance residuals and then the weighted residual sum of squares

$$( WRSS ) ( = {\sum }_{i = 1}^{n} {w}_{i} {r}_{i}^{2} )$$

where rᵢ represents the deviance residual for the ith observation. In addition, the final fit may be checked against a diagnostic plot based on a result under the Cox proportional hazard model transformed to an exponential hazard function. The candidate statistical model that best fits the non-genetic data, as weighted using weights wᵢ may then be used as a prediction model to predict the risk of disease for the subject of interest This prediction model is preferably a function of the non-genetic indicators, and should yield statistically valid results for subjects having genetic indicators identical to those of the candidate subject.

For example, as mentioned earlier, the prediction model 116 may be a survival function for individuals having a particular set of genetic markers, where the independent variables of the function are non-genetic indicators such as age and body mass index. A survival function is a function for calculating the probability that an event, such as acquiring a disease, has yet to happen at a given time. As is apparent, only a fraction of sampled data (e.g. non-genetic data) needs to be analyzed (e.g., fitted) during this final optimization step S208.

Analysis of the non-genetic data 112 may be carried out using any known statistical techniques. For example, the non-genetic data 112 may be fit to any suitable survival models, such as a Cox proportional hazard regression model described above. Non-genetic data 112 may also be fit to more than one family of models. The candidate models 708 and the fitting results may be evaluated or compared to arrive at a model that best describes the non-genetic data 112. During the evaluation or comparison, data in each data set 104 may be weighed by its corresponding weight 114 obtained in step S206 in any suitable manner.

Steps S204 to S208 may be iterated so that both the group weights and the parameters may be optimized for a given family of candidate models 708. For example, the group weights 510 may be adjusted to find a global minimum of the weighted sum of deviates 712. It is possible that for different families of candidate models 708, different sets of group weights 510 may result for a given reference group 508. In such cases, the global minimums of the weighted sum of deviates 712 for different model families may be compared to arrive at the best model, which is then taken as the prediction model 116.

In step S210, a disease risk for patient 120 (FIG. 1) of population 108 who has the particular combination of genetic data may be calculated using the prediction model 116 determined with non-genetic data of the patient 120 as input. Using the above mentioned survival function as an example, the non-genetic indicators may be input into the survival function to arrive at a new function S (t) with only age as the independent variable. If one defines a cumulative disease risk R (t) as being the probability of the member having acquired the disease at age of t given its genetic and non-genetic data, then R (t)=1−S (t). The disease risk of the member at any given age can therefore be calculated from R (t).

As can be appreciated, by using this method it is possible to analyze genetic data and non-genetic data separately, without having to directly untangle the interwoven and intractable relationship between them, and yet not ignoring the effects of either. Also, it is possible to significantly reduce the amount of computation in the case of a large number of risk factors, as only data indicative of a subset of the risk factors is analyzed at a time.

As can be appreciated, using steps detailed in FIGS. 2-7 embodiments of the present invention may determine a statistical risk prediction model 116 for each reference group 508, which corresponds to one particular combination of genetic markers, instead of determining one model all possible combinations of genetic markers. Yet, the risk prediction model 116 is determined by taking into account of all available data, including that in data sets of different genetic combinations. Thus, the genetic data 110 of every genetic indicator is included in the analysis, meaning that the risk prediction model 116 reflects the effects of all genetic factors. However, because only non-genetic data 112 is fit to one or more candidate models 708, the computation is significantly less intensive than what would be required for fitting both genetic and non-genetic data together, particularly when there is a large number of genetic indicators. Further, because the analysis (fitting) does not attempt to unravel the intractable interplay and interaction between genetic and non-genetic factors directly in one fit, consistent and reliable results may be obtained.

As will be understood by a person skilled in the art, whenever intensive computation is required, the calculations can be carried out in a distributed or parallel manner. Specifically, the computer 100 may communicate with one or more processing units or other computers through a network (not illustrate). This network may be a local area network, a wide area network, an intranet, the Internet, wireless networks, and the like. The networked processing units or computers may each carry out only part of the calculations. For example, as alluded to earlier, the imputation of missing data and the calculation of corresponding weights 114 (including both group weights 510 (FIG. 5) and sampling weights) can be computationally intensive and may be carried out in a distributed or parallel manner. For instance, missing data in different batches 416 (FIG. 4) may be imputed by different processing units or computers. Group weights 510 for different reference groups 508 may be determined by different processing units or computers. Further, when a large number of risk curves are to be calculated, steps S206 and S208 can also be carried out in a distributed or parallel manner. For instance, one of the computers may calculate risk curves for members belonging to a particular reference group 508. The calculations may be orchestrated by computer 100 or another computer in communication with computer 100. Data sets 104 may be stored on a storage connected to the network. Computer 100 may transmit to each of the other computers the information necessary for each computer to carry out its assigned calculations and receive the results from each of the other computers after the calculations have been completed.

In the above description, the risk prediction model 116 is determined by partitioning the genetic data 110 and fitting the non-genetic data 112 for different combinations of genetic markers. This approach is consistent with the underlying etiological conjecture that an individual's genetic makeup determines the baseline disease risk, which is modified by environmental factors. However, there may be situations where it is desirable to partition the non-genetic data 112 and fit the genetic data 110 for different combinations of non-genetic data 112. It is also possible to partition only part of the genetic or non-genetic data, or even a mixture of both.

Further, it is not necessary to use all data sets 104 in each step of a method embodying the present invention. It is also not necessary that only data sets 104 are used in every step. For example, data sets 104 may be partitioned based on prior knowledge, meaning that the data sets used to arrive at the partition tree 600 (FIG. 6) need not be the same as the data sets 104 used for determining the group weights 510 and the risk prediction model 116. The partition tree may also be build using only a part of the data sets 104. Once a partition tree 600 is built, the combination of genetic markers for each terminal node 506 is known. Data sets 104 may be partitioned according to the combination of genetic markers of the terminal nodes 506 without additional further data analysis such as building a new tree 600. Likewise, the group weights 510 may also be obtained from prior analysis of other data sets, or analysis of part of data sets 104.

As can be appreciated from the description herein and the figures, the embodiments of the present invention are effective and efficient in analyzing a large number of factors, both genetic and non-genetic, that affect a disease risk. The embodiments of the present invention also make efficient use of available data and computing resources.

Described next is an exemplary risk predicting system embodying the present invention. The particular embodiment is known as a Complex Disease Risk Assessment System (CD-RAS), and more specifically, a Coronary Artery Disease Risk Assessment (CADRA) system, which employs a Genetic Risk Assessment Tree (GRAT) model for predicting complex disease risks for coronary artery disease (CAD).

CAD causes an estimated death toll of 50 million per year worldwide and is the leading cause of morbidity and premature mortality in developed and developing countries. CAD has many etiological factors, both genetic and environmental. The heritability of CAD is estimated at 65%, meaning genetic factors' contribution to the risk of the disease is 65%. The process of artery blockage starts as early as in the eighth month of life. Thus, knowledge of the effects of both genetic and environmental risk factors would be particularly useful in preventing or significantly delaying the disease by modifying the relevant environmental factors sufficiently early in life. There is currently no genetic testing of CAD because the contribution from each individual susceptibility gene to the risk is small.

An example analysis is performed with the CADRA system, using 32 genetic markers and seven non-genetic factors for the population 108 of Singapore.

The genetic markers chosen are polymorphic sites found on CAD susceptibility genes that are related to lipid metabolism, blood coagulation and blood pressure regulation and etc.

The seven non-genetic indicators used were age, sex, race, body mass index, smoking status, medical history of diabetes mellitus, and family history of diabetes mellitus.

Demographic information and health statistics were obtained from the Ministry of Health, Singapore.

In step S202, clinical data 310 is partially drawn from the CADRA database (S306). The data sets 104 are taken from 2949 subjects, of whom 1426 are sick subjects and 1487 are healthy subjects.

The sick subjects 106 were consecutive patients who had been admitted to hospital for coronary artery bypass graft surgery. Blood was collected during pre-operative review and at least three months after full recovery from those with a history of myocardial infarction. The inclusion criterion was at least 50% stenosis in one or more of the major coronary arteries.

The healthy subjects 106 were selected from individuals undergoing routine annual medical examinations offered by their employers. Physical examinations and laboratory tests such as blood hemoglobin estimation, urine analysis for albumin and sugar, chest X-ray and resting electrocardiogram were carried out.

Genomic DNA was prepared from blood samples according to the method of Parzer. Polymerase chain reaction (PCR) was carried out in reaction mixtures containing 1 μM of primers, 200 μM of dNTPs, 2% of DMSO, 0.01 u/μl of DNA polymerase (Qiagen, Germany) in 50 μl of the reaction buffer. The temperature profile for most of the PCR reaction was typically three minutes at 93° C. for the first denaturation step, followed by one minute at 93° C., one minute at 55° C., one minute at 72° C. for 35 cycles, and 10 minute for the last extension at 72° C.

Genotyping was carried out by a chip-based method as described by Syvanen, which allows all polymorphisms be genotyped simultaneously.

In step S308, missing data was imputed as follows:
1) calculating the correlation matrix for the 32 genetic markers (S402);
2) grouping genetic markers into 13 batches 410 of correlated genetic markers by factor analysis (S404);
3) determining non-genetic indicators related to each batch 410 (S406);
4) grouping data sets 104 into batches 416 consisting of correlated genetic data 110 and non-genetic data 112 and imputing missing data in each batch 416 separately (S408).

In step 204, Cox models were selected for analyzing the data.

In steps 206, the corresponding weights 114 were determined as follows.

First, adjustment factors were determined based on the combined demographic and health statistical data for the Singapore population, using equation (1) as described above. The following characteristics were used: gender, race, age, body mass index, smoking, hypertension, cholesterol, and family history.

Next, in step S502, the data sets 104 were partitioned using the 32 genetic markers with the GRAT model (tree 600), depending on the presence and absence of each genetic marker. One criterion for splitting a node 602 was the deviance of fitting. Another criterion was the minimum group size, which was set at 50.

A tree-pruning step was carried out after the tree was built, using a likelihood ratio test. The ratio of likelihood before and after a split (LR) was calculated as:

$$LR = \frac{L ( parent\_group )}{L ( subgroup1 ) * L ( subgroup2 )} .$$

where a likelihood (L) was calculated as:

$$L = {\prod }_{i = 1}^{n} {f ( {t}_{i} {\vert x )}^{{δ}_{i}} S ( {t}_{i} \vert x )}^{1 - {δ}_{i}} ,$$

where f( ) was the probability function of the CAD event given x and S( ) was the survival function given x. “x” represents the non-genetic variables. δ₁ equals 0 for healthy subjects and 1 for CAD subjects. In terms of the hazard function and the risk function

$$L = {\prod }_{i = 1}^{n} \frac{{h ( {t}_{i} \vert x )}^{{δ}_{i}}}{1 - R ( {t}_{i} \vert x )} 1 - {R ( {t}_{i} \vert x )}^{1 - {δ}_{i}} .$$

To pass the test, the value −2log(LR) must be greater than the 95^{th} percentile of a χ² distribution. In this example, the data is eventually partitioned into 13 groups.

In step S504, the group weights 510 were determined as follows:

The reference group 508 was always assigned a group weight of one (gᵢᵢ=1). Other group weights with respect to the reference group were optimized simultaneously by minimizing the sum of squared residuals for the data in the reference group. The optimization routine included a 10-fold cross-validation procedure, as described below for, e.g., group G₁,
(1) Set the initial values of the group weights as g₁₁=1, g₁₂= . . . =g_{1G}=0.5, where 0≦g₁ᵢ≦1. Obtain the corresponding weight for each data set by multiplying its group weight and adjustment factor, wᵢ=aᵢ×g₁ᵢ.
(2) Calculate total residuals for G₁ with the given set of corresponding weights, using tenfold cross-validation. The target function was

$$f ( \{ {g}_{1 i} \} ) = {\sum }_{1}^{{n}_{1}} {w}_{i} {r}_{i , cv}^{2} ,$$
where r_{i,cv}² represented the squared deviance residual. The tenfold cross-validation procedure was carried out as follows:
   (I) Randomly divide G₁ into 10 subgroups at random S_{1,1}, . . . , S_{1,10}.
   (II) For S_{1,1}, fit data indicative of non-genetic factors and disease status in all data sets except those in S_{1,1} to the Cox model. This produces an (local) optimal set of coefficients for the given set of corresponding weights.
   (Ill) With the coefficients determined in (II), calculate the sum of residuals for all data sets in subgroup S_{1,1}.
   (IV) Repeat (II) and (IIl) for each of the 10 subgroups. The residual for G₁ is the sum of all residuals for all 10 subgroups.
(3) The optimal groups weights with reference to G1 were determined by minimizing the total residual for G₁ as calculated in (2).

The above steps [(1) to (3)] were repeated for all reference groups. A total of 13 sets of group weights were determined.

The corresponding weight 114 for each data set 104 was calculated as the product of its associated adjustment factor and optimal group weight.

In step S208, the corresponding weights 114 with reference to group Gᵢ were used to optimize the coefficients of the hazard function for Gᵢ by fitting all data sets to the Cox model. Thus, a total of 13 sets of optimal coefficients were determined, one set for each group.

Steps S204 to S208 were repeated with Cox models having different numbers of coefficients in the hazard function. The particular function that produced the best overall result was used in the final model.

The resulting prediction models 116 were used to calculate the disease risk for patients that fall within the respective reference group.

The results as obtained above were evaluated based on two different methods of classification of the subjects.
(1) The first classification method classifies the subjects as “at risk” versus “not at risk.” Subjects at risk are those whose risk of the disease is higher than a threshold C. That is, a subject is at risk if R>C, not at risk if R≦C. The threshold C is calculated from the data to optimize the sensitivity and specificity of the method.
(2) The second method classifies the subjects as at high, medium, or low risk. There are two thresholds: H and L. A subject is at high risk if R>H, medium risk if L<R≦H, low risk if R≦L. The thresholds H and L are chosen as follows: H is chosen to cover the upper two-thirds of the subjects at risk, and L is chosen to cover the lower two thirds of the subjects not at risk. As such, the medium risk group would always comprise 33% of the subjects.

The results are listed in Table 1. It is shown that the percentage of subjects who had CAD but who are predicted at low risk is only 3%, whereas the percentage of subjects without CAD that were found to be at high risk is 12%.

TABLE 1
Results of the GRAT Model with 10-fold cross-validation.

| | Healthy | CAD | Risk |
|---|---|---|---|
| Stratified by At Risk | | | |
| and Not at Risk | | | |
| Subjects | 1487 | 1426 | |
| Not at Risk | 1129 (76%) | 167 (11%) | R > 5.6% |
| At Risk | 358 (24%) | 1295 (89%) | R ≦ 5.6% |
| Stratified by High, | | | |
| Medium and Low Risk | | | |
| Low Risk | 822 (55%) | 42 (3%) | R ≦ 0.8% |
| Med Risk | 483 (33%) | 500 (34%) | 0.8% < R ≦ 30% |
| High Risk | 182 (12%) | 920 (63%) | R > 30% |

The results of this risk prediction model are about 83% correct on average. Sensitivity of the test is 89% and specificity is 76%. The calculations indicate that body mass index did not have a strong contribution to risk of CAD. The calculations also show that hypertension and diabetes are both strongly correlated to personal or family history. Since each pair contributes equally to the risk of CAD and are strongly correlated, only personal and family history of diabetes mellitus were used as risk factors in the final model in order to reduce variable factors. Among the 32 genetic markers, 17 markers are shown to significantly contribute to the prediction of risk of CAD, demonstrating that the CADRA system is able to recognize genetic markers that are good predictors of CAD disease.

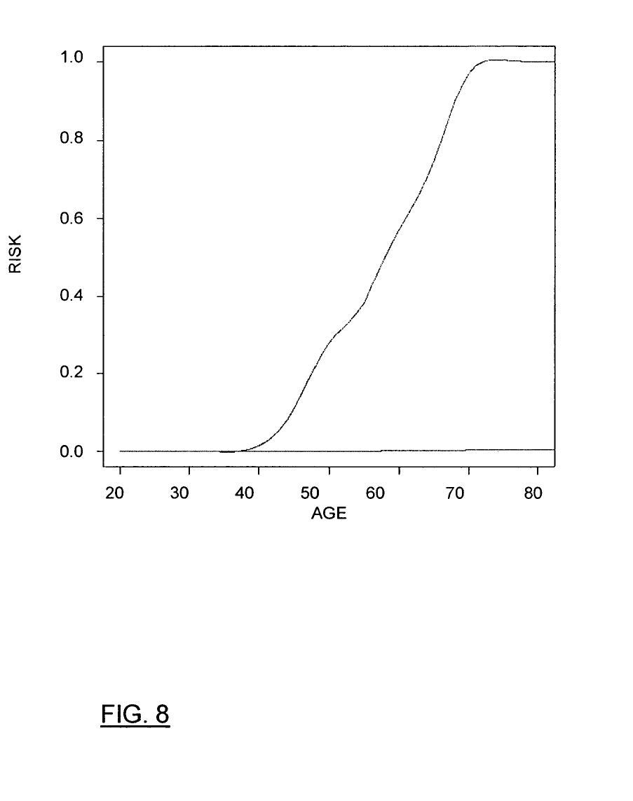

Two example risk curves from the above calculations are shown in FIG. 8. The relevant subjects are two Chinese females with similar non-genetic data 112 but different genetic data 110. Both subjects have no medical history. As illustrated, the two risk curves are very different even though the subjects share similar non-genetic data. This result illustrates that risk of CAD is strongly influenced by genetic factors.

The aforementioned and other features, benefits and advantages of the present invention can be understood from this description and the drawings by those skilled in the art.

Although only a few exemplary embodiments of this invention have been described above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

1. A method of determining a statistical model for predicting disease risk for a member of a population,
a. collecting a plurality of sets of data, each of said sets of data associated with one member of said population, and comprising data of a first type, data of a second type, and an indicator of disease status of said one member associated with said set;
b. selecting a candidate statistical model for calculating said disease risk as a function of data of said first type, said candidate model dependent on a plurality of parameters;
c. determining a plurality of weights, each one of said weights associated with one of said sets of data and indicating a statistical significance of said one of said sets of data, wherein weights associated with sets of said data having like data of said second type are the same; and
d. optimizing said parameters of said candidate model by fitting said plurality of sets of data to said candidate model, taking into account said weights.

2. The method of claim 1, wherein data of said first type is non-genetic data and data of said second type is genetic data.

3. The method of claim 1, wherein said corresponding weights are used to assess a goodness of said fitting.

4. The method of claim 1, wherein said determining comprises:
a. grouping said collected data into groups such that all sets of data within each said group have like data of said second type, one of said groups being a reference group which contains sets of data having data of said second type like data of said second type obtained from said member of said population; and
b. determining a group weight for each said group, whereby said group weight is the corresponding weight for each set of data within said each group.

5. The method of claim 4, wherein the group weight of said reference group has a value of one and each of the other group weights has a value between zero and one.

6. The method of claim 5, wherein said other group weights are optimized by minimizing a target function, said target function dependent on a plurality of residuals, one of said residuals for each of the data sets in said reference group.

7. The method of claim 6, wherein a residual for the Ah one of said data sets is the difference between the value of the indicator of disease status contained in said ith data set and the value of disease risk for the member associated with said ith data set, said value of disease risk calculated from said candidate model with said parameters optimized for a given set of group weights by fitting data sets in groups other than the reference group to said candidate model.

8. The method of claim 7, wherein said target function is of the form:
ƒ=Σwᵢφ(rᵢ),
where
wᵢ is the corresponding weight for data set i; and
rᵢ is the residual for data set i.

9. The method of claim 1, wherein data of said first type comprises data indicative of time.

10. The method of claim 9, wherein said candidate model is a Cox proportional hazard regression model.

11. The method of claim 9, wherein said candidate model is a disease risk function of the form:
$$R ( t ) = 1 - exp \{ - {\int }_{0}^{t} h ( u ) ⅆ u \} ,$$
where
R(t) represents said disease risk at a given time t;
h(u) is of the form:
$$h ( u ) = {h}_{0} ( u ) exp ( {\sum }_{1}^{{n}_{c}} {β}_{i} {x}_{i} ) ;$$
h₀(u) is dependent only on u;
xᵢ is a variable indicative of a disease risk factor, said collected data containing a plurality of values of xᵢ;
βᵢ is a coefficient for xᵢ and
n_{c} is the number of coefficients in said disease risk function.

12. The method of claim 1, wherein said collecting comprises imputing missing data to said plurality of data sets.

13. The method of claim 1, wherein each corresponding weight is weighted by an adjustment factor indicative of the representativeness of the member associated with said each corresponding weight.

14. The method of claim 13, wherein an adjustment factor aᵢ for a data set obtained from a member i of said population is calculated as:
$${a}_{i} = \frac{{n}_{i}^{p}}{{n}_{i}^{s}} ,$$
where nᵢ^{p} is the number of members in said population who share a same set of characteristics with said member i, and nᵢ^{s} is the number of members associated with said collected data who share said set of characteristics.

15. The method of claim 14, wherein said set of characteristics comprises non-genetic factors.

16. The method of claim 14, wherein said set of characteristics comprises genetic factors.

17. The method of claim 14, wherein said set of characteristics comprises both genetic and non-genetic factors.

18. The method of claim 14, wherein said set of characteristics are selected from the group of age, gender, race, body mass index, smoking status, hypertension, cholesterol level, personal health history, and family health history.

19. The method of claim 1, comprising calculating a disease risk for said member of said population with said disease risk prediction model.

20. A computing system adapted for perform the method of any one of claims 1 to 19.

21. An article of manufacture comprising
a computer readable medium embedded thereon computer executable instructions, which when executed by a computer causes said computer to determine a statistical model for predicting disease risk for a member of a population by
a. collecting a plurality of sets of data, each of said sets of data associated with one member of said population, and comprising data of a first type, data of a second type, and an indicator of disease status of said one member associated with said set;
b. selecting a candidate statistical model for calculating said disease risk as a function of data of said first type, said candidate model dependent on a plurality of parameters;
c. determining a plurality of weights, each one of said weights associated with one of said sets of data and indicating a statistical significance of said one of said sets of data, wherein weights associated with sets of said data having like data of said second type are the same; and
d. optimizing said parameters of said candidate model by fitting said plurality of sets of data to said candidate model, taking into account said weights.

22. A method of imputing missing data indicative of a plurality of factors, comprising:
a. determining a correlation between said plurality of factors;
b. grouping said factors into batches such that all factors in each said batch are correlated; and
c. imputing missing data for factors in one said batch at a time.

23. A method of grouping a plurality of data sets into groups, comprising:
a. dividing said plurality of data sets into two or more groups depending on data indicative of a factor of a first type in each of said data sets;
b. determining if a criterion is met after said dividing, said criterion is evaluated based on data of a second type in each of said data sets; and
c. when said criterion is not met, regrouping said plurality of data sets back into one group.

24. The method of claim 23, wherein said dividing is performed recursively on each group of a division.

25. The method of claim 24, wherein divisions at different levels are made dependent on data indicative of different factors.

26. The method of claim 25, wherein a branch of said recursive division is terminated at the level at which said criterion is not met.

27. A method of weighing a plurality of data sets, each one of said data sets associated with a member of a population, comprising:
weighing each set of said plurality of data sets by a weight indicative of the representativeness of the member associated with said each set, wherein a weight aᵢ for a data set obtained from a member i of said population is calculated as:
$${a}_{i} = \frac{{n}_{i}^{p}}{{n}_{i}^{s}} ,$$
where nᵢ^{p} is the number of members in said population who share a same set of characteristics with said member i, and nᵢ^{s} is the number of members associated with said collected data who share said set of characteristics.
